(19) United States
(12) Patent Application Publication
(10) Pub. No.: US 2023/0405315 A1
(43) Pub. Date: Dec. 21, 2023

(54) Compositions And Methods For Treating With A Combination Of Alternating Electric Fields And FGF Inhibitors

(71) Applicant: NOVOCURE GMBH, Root D4 (CH)

(72) Inventors: Tali Voloshin-Sela, Haifa (IL); Lilach Avigdor, Haifa (IL); Anat Klein-Goldberg, Haifa (IL); Rom Paz, Haifa (IL); Elizabeth Moyal, Toulouse (FR); Valerie Gouaze Andersson, Toulouse (FR)

(21) Appl. No.: 18/336,880

(22) Filed: Jun. 16, 2023

Related U.S. Application Data
(60) Provisional application No. 63/353,559, filed on Jun. 18, 2022.
(60) Provisional application No. 63/499,842, filed on May 3, 2023.
(60) Provisional application No. 63/504,093, filed on May 24, 2023.

(51) Int. Cl. A61N 1/36; A61P 35/00; A61K 41/00

(52) U.S. Cl. CPC A61N 1/36002; A61K 41/0023; A61P 35/00

(57) ABSTRACT

Disclosed are methods of treating a subject in need thereof comprising: applying alternating electric fields, at a frequency for a period of time, to a target site of the subject in need thereof; and administering one or more FGF inhibitors or FGFR inhibitors to the subject in need thereof. Disclosed are methods of increasing a cell's sensitivity to alternating electric fields comprising: applying alternating electric fields, at a frequency for a period of time, to a cell; and contacting one or more FGF inhibitors or FGFR inhibitors to the cell, thereby increasing the cell's sensitivity to the alternating electric fields. Disclosed are methods of increasing cytotoxicity in a cell comprising: applying alternating electric fields, at a frequency for a period of time, to a cell; and contacting one or more FGF inhibitors or FGFR inhibitors to the cell, thereby increasing cytotoxicity in the cell. Disclosed are methods of maintaining, or enhancing, sensitivity to alternating electrical fields in a cell comprising: applying alternating electric fields, at a frequency for a period of time, to a cell; and contacting one or more FGF inhibitors or FGFR inhibitors to the cell, thereby maintaining, or enhancing, sensitivity to the alternating electrical fields in the cell. In some aspects, maintaining, or enhancing, sensitivity of a cell to alternating electrical fields is the same as reducing resistance of the cell to alternating electrical fields.

### CROSS-REFERENCE TO RELATED APPLICATIONS

This application claims the benefit of U.S. Provisional Patent Application Nos. 63/353,559, filed Jun. 18, 2022, 63/499,842, filed May 3, 2023, and 63/504,093, filed May 24, 2023, each of which is incorporated by reference herein in its entirety.

### BACKGROUND

Fibroblast growth factor (FGF) signaling pathway regulates numerous cellular processes such as cell proliferation, apoptosis, angiogenesis, migration, invasion and metastasis. FGF/FGF receptor (FGFR) exhibits its physiological functions via regulation of its downstream targets. The chemical inhibitors of FGF/FGFR, antibodies and natural agents can be used to block the FGF signaling pathway. Thus, targeting FGF/FGFR could be an effective approach for the treatment of cancer patients in combination with tumor treating fields (TTFields).

### BRIEF SUMMARY

Disclosed are methods of treating a subject in need thereof comprising: applying alternating electric fields, at a frequency for a period of time, to a target site of the subject in need thereof; and administering one or more FGF inhibitors or FGFR inhibitors to the subject in need thereof.

Disclosed are methods of increasing a cell's sensitivity to alternating electric fields comprising: applying alternating electric fields, at a frequency for a period of time, to a cell; and contacting one or more FGF inhibitors or FGFR inhibitors to the cell, thereby increasing the cell's sensitivity to the alternating electric fields.

Disclosed are methods of increasing cytotoxicity in a cell comprising: applying alternating electric fields, at a frequency for a period of time, to a cell; and contacting one or more FGF inhibitors or FGFR inhibitors to the cell, thereby increasing cytotoxicity in the cell.

Disclosed are methods of maintaining, or enhancing, sensitivity to alternating electrical fields in a cell comprising: applying alternating electric fields, at a frequency for a period of time, to a cell; and contacting one or more FGF inhibitors or FGFR inhibitors to the cell, thereby maintaining, or enhancing, sensitivity to the alternating electrical fields in the cell. In some aspects, maintaining, or enhancing, sensitivity of a cell to alternating electrical fields is the same as reducing resistance of the cell to alternating electrical fields.

Additional advantages of the disclosed methods and compositions will be set forth in part in the description which follows, and in part will be understood from the description, or may be learned by practice of the disclosed method and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed methods and compositions.

FIG. 1 is a block diagram of a system for applying an alternating electric field.

FIG. 2 shows a characterization of secretory cytokines following TTFields application using human cytokine array assay. A cytotoxic effect was observed after 3 days of TTFields treatment in optimum cytotoxic frequencies. A2780 and H1299 cell count relative to control of cells after TTFields application for 3 days in optimal cytotoxic frequencies 200 kHz and 150 kHz respectively.

FIGS. 3A and 3B show cytokine array results of an upregulation of FGF-19, FGF-7 and FGF-basic expression following 3 days of TTFields application in vitro in (FIG. 3A) A2780 cell line, TTFields frequency 200 kHz; and (FIG. 3B) H1229 cell line, TTFields frequency 150 kHz. Images of spots are shown from the Human Cytokine Array membranes of TTFields Treated cells and Control group of all three FGF targets: FGF basic, FGF 7 and FGF 19. Quantification of pixel density from the cytokine array membranes are represented in the graphs. The graphs comprise an average of two biological repeats for each target.

FIGS. 4A and 4B show mouse cytokine array results from in vivo experiments as validation for FGF21 upregulation. (FIG. 4A) In vivo ovarian cancer model experiment design. Female Mice were treated with TTFields for 10 days vs. the control sham heat group. (FIG. 4B) Images of spots from the mouse cytokine array membranes TTFields treated mice and control sham heat mice. The graph shows quantification of FGF21 pixel density from the cytokine array membranes spots.

FIG. 5 shows a response to TTFields in different glioblastoma stem cell (GSC) cell lines. A cytotoxic effect was seen on 6 GBM biopsy specimen (SRA4,SRC1, SRC2,SRB2, SRAS, and SRB3) was observed after 3 days of TTFields treatment at frequency 200 kHz. Bars represent the mean±SD of three different experiments.

FIG. 6 shows increased mRNA expression of FGFR1 following TTFields application. FGFR1 mRNA level was quantified by qPCR in SRC1 and SRC2 glioblastoma stem cells after 3 days treatment with TTFields (200 kHz). Results are expressed in Fold change of FGFR1 expression in cells treated with TTFields compared to FGFR1 expression in non treated cells with TTFields.

FIG. 7 shows that TTFields application induces mRNA expression of FGFR1 that is inhibited by addition of FGFR1 inhibitor. Pemigatininb (PMGB) is an FGFR inhibitor. SRC1 and SRC2 were treated concomitantly with PMGB (125 nM) and TTFields (200 kHz) for 3 days. Results are expressed in Fold change of FGFR1 expression in cells treated with TTFields compared to FGFR1 expression in control cells.

FIG. 8 shows cell survival to TTFields in the presence of FGFR1 inhibitor. A cytotoxic effect on 5 GBM biopsy specimen (SRA4, SRC1, SRC2, SRB2, and SRA5) was observed after 3 days of TTFields (200 kHz) and PMGB (125 nM) treatment.

FIG. 9 shows cell survival to TTFields in the presence of FGFR1 inhibitor. A cytotoxic effect on 5 GBM biopsy specimen (SRA4, SRC1, SRC2, SRB2, and SRA5) was observed after 3 days of TTFields (200 kHz) and PMGB (125 nM) treatment.

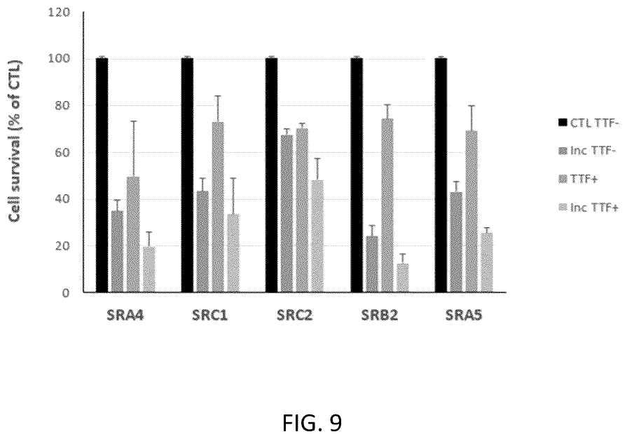

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a FGF inhibitor” or “a FGFR inhibitor” includes a plurality of such inhibitors, reference to “the inhibitor” is a reference to one or more inhibitors and equivalents thereof known to those skilled in the art, and so forth.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the brain. In some aspects, a cell or population of cells that can be a target site or a target cell include, but are not limited to, a cancer cell (e.g., an ovarian cancer cell). In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g., a target site such as a cell). In some aspects, the alternating electrical field can be in a single direction or multiple directional. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and is described in U.S. Pat. No. 7,565,205, which is incorporated herein by reference in its entirety for its teaching of TTFields.

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field. For example, the subject of administration can be a subject with ovarian cancer or lung cancer.

By “treat” is meant to administer or apply a therapeutic, such as alternating electric fields and a vector, to a subject, such as a human or other mammal (for example, an animal model), that has cancer or has an increased susceptibility for developing cancer, in order to prevent or delay a worsening of the effects of the disease or infection, or to partially or fully reverse the effects of cancer. For example, treating a subject having glioblastoma can comprise delivering a therapeutic to a cell in the subject.

By “prevent” is meant to minimize or decrease the chance that a subject develops cancer.

As used herein, the terms “administering” and “administration” refer to any method of providing a FGF or FGFR inhibitor to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat cancer. In further various aspects, a preparation can be administered prophylactically; that is, administered for prevention of cancer. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

B. Alternating Electric Fields

The methods disclosed herein comprise alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 kHz and 1 MHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be electric fields at 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the field strength of the alternating electric fields can be between 0.5 and 4 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more of consecutive exposure. In some aspects, the exposure may be for at least 6 hours, at least 12 hours, at least 24 hours, at least 36 hours, at least 48 hours, or at least 72 hours or more of cumulative hours of exposure. For example, in some aspects, the exposure can occur at different times over a period of days, weeks, or months. In some aspects, the patient is applying the alternating electric field at least 50%, 60%, 70%, 80%, or 90% of the time of treatment.

In some aspects, application of the alternating electric fields can be continuous or discontinuous. For example, in some aspects, short breaks of application of the alternating electric fields can occur to allow the subject to shower or otherwise have a rest from therapy. In some aspects, regardless of any short breaks of application of the alternating electric fields, subjects receive alternating electric fields for at least 50%, 60%, 70%, 80%, 90% of treatment time.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

C. FGF/FGFR Inhibitors

As used herein, FGF inhibitors are compositions that bind to an FGF protein, peptide or nucleic acid that encodes an FGF and prevents the FGF from interacting with or binding with one or more FGFRs. In some aspects, the FGF inhibitor specifically inhibits or decreases FGF expression. In some aspects, the FGF inhibitor specifically binds to a nucleic acid that encodes an FGF. In some aspects, “specifically” means that the inhibitor is selective to FGF or FGFR as opposed to a non-selective inhibitor which may target multiple receptors. For example, in some aspects the disclosed FGF or FGFR inhibitors are selective to FGF or FGFR and do not also inhibit a second receptor, such as VEGFR. In some aspects, an inhibitor that specifically inhibits FGF or FGFR can have an IC50 less than 30 nM, less than 20 nM, less than 10 nM, less than 5 nM, less than 1 nM against at least one of FGF1, 2, 3, or 4 or FGFR1, 2, 3, or 4. For example, Pemigatinib has a significantly lower IC50 against at least one of FGF1, 2, 3, or 4 than lenvatinib (nonselective inhibitor of FGFR) (Kommalapati et al. Cancers. 2021 June 13; 13(12):2968)

In some aspects, the FGF is FGF-21, FGF-19, FGF-7, or FGF-basic. In some aspects, the FGF inhibitor can be a small molecule, peptide, protein, antibody, or nucleic acid (e.g. siRNA). Examples of FGF inhibitors include, but are not limited to, soluble FGFR, such as soluble FGFR3, and soluble decoy receptors, such as FGF-Trap that is a soluble decoy receptor fusion protein that binds FGF-2.

As used herein, FGFR inhibitors are compositions that bind to an FGFR protein, peptide or nucleic acid that encodes an FGFR and prevents the FGFR from interacting with or binding with one or more FGFs. In some aspects, the FGFR inhibitor specifically inhibits or decreases FGFR expression. In some aspects, the FGFR inhibitor specifically binds to a nucleic acid that encodes an FGFR. In some aspects, the FGFR is FGFR1, FGFR2, FGFR3, or FGFR4. In some aspects, the FGFR inhibitor can be a small molecule, peptide, protein, antibody, or nucleic acid (e.g. siRNA). In some aspects, the FGFR inhibitor can be one or more of the inhibitors listed in Table 1.

TABLE 1
Examples of FGFR inhibitors.

| Compound | Target |
|---|---|
| BAY 1179470 | FGFR2 |
| FPA144 | FGFR2 |
| PRO-001 | FGFR3 |
| RG7444 | FGFR3 |
| SSR128129E | FGFRs |
| AZD4547 | FGFR1-3 |
| BAY1163877 | FGFRs |
| BGJ398 | FGFR1-3 |
| CH5183284 | FGFR1-3 |
| Erdafitinib | FGFRs |
| LY2874455 | FGFRs |
| Roblitinib | FGFR4 |
| Infigratinib | selective FGFR inhibitor |
| (BGJ398) | for FGFR1/2/3 |
| SSR128129E | FGFR1 |
| PD-166866 | FGFR1 |
| ASP5878 | FGFR1, 2, 3, and 4, |
| H3B-6527 | FGFR4 |
| NSC12 | FGF2/FGFR |
| BO-264 | blocks the function of |
| | FGFR3-TACC3 |
| Fisogatinib | FGFR1/4 |
| (BLU-554) | |
| FIIN-2 | Pan-FGFR |
| Futibatinib | FGFR1/2/3/4 |
| (TAS-120) | |
| BLU9931 | FGFR4 |
| Pemigatinib | FGFR1/2/3/4 |
| (INCB054828) | |
| Zoligratinib | FGFR1/2/3/4 |
| (Debio-1347) | |
| Alofanib | FGFR2 |
| (RPT835) | |
| PRN1371 | FGFR1/2/3/4 |
| Ferulic Acid | FGFR1/2 |
| Derazantinib | FGFR1/2/3/4 |
| (ARQ-087) | |

D. Compositions

Disclosed are compositions and formulations comprising one or more FGF inhibitors or FGFR inhibitors, or a combination thereof. In some embodiments the formulation further includes a pharmaceutically acceptable carrier or diluent. For example, disclosed are pharmaceutical compositions, comprising an FGF inhibitor or FGFR inhibitor and a pharmaceutically acceptable carrier. For example, disclosed are pharmaceutical compositions, comprising pemigatinib or AZD4547, and a pharmaceutically acceptable carrier. Disclosed also are pharmaceutical compositions, comprising a FGF inhibitor or FGFR inhibitor and a pharmaceutically acceptable diluent.

In some aspects, the FGF inhibitor or FGFR inhibitor can be administered with a pharmaceutically acceptable carrier and/or diluent in any of the disclosed methods.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidylcholine (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. In the methods described herein, delivery of the disclosed compositions to cells can be via a variety of mechanisms. The pharmaceutical composition may be administered in a number of ways depending on whether local or systemic treatment is desired, and on the area to be treated.

1. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

E. Methods of Treating

Disclosed are methods of treating a subject in need thereof comprising: applying alternating electric fields, at a frequency for a period of time, to a target site of the subject in need thereof; and administering one or more FGF inhibitors or FGFR inhibitors to the subject in need thereof. Also disclosed are methods of treating a subject in need thereof comprising: applying alternating electric fields, at a frequency for a period of time, to a target site of the subject in need thereof; and administering a composition comprising one or more FGF inhibitors or FGFR inhibitors to the subject in need thereof.

In some aspects, the subject in need thereof has cancer. In some aspects, the subject in need thereof has mesothelioma, ovarian cancer, or lung cancer (e.g., non-small cell lung cancer). In some aspects, the subject in need thereof has brain cancer (e.g., glioblastoma cells), pancreatic cancer, breast cancer, liver cancer, or colon cancer.

In some aspects, the target site comprises one or more cancer cells. In some aspects, the target site comprises one or more mesothelioma cells, ovarian cancer cells, lung cancer cells (e.g., non-small cell lung cancer cells), brain cancer cells (e.g., glioblastoma cells), pancreatic cancer cells, breast cancer cells, liver cancer cells, or colon cancer cells.

In some aspects, the alternating electric fields are applied before, after, or simultaneously with administering the one or more FGF inhibitors or FGFR inhibitors. In some aspects, the step of applying the alternating electric fields begins at least one hour before a FGF inhibitor or FGFR inhibitor. In some aspects, the step of applying the alternating electric fields begins at least 30 minutes before a FGF inhibitor or FGFR inhibitor. In some aspects, applying the alternating electric fields simultaneously can mean applying within 5, 10, 15, 20, 25, 30, 35, 45, 50, 55, or 60 minutes before or after administering a FGF inhibitor or FGFR inhibitor. In some aspects, the alternating electric fields can be applied and the FGF inhibitor or FGFR inhibitor administered at least 1, 2, 3, 4, 5, 6, 7, 8, 9, 10, 11, 12, 13, 14, 15, 16, 17, 18, 19, 20, 21, 22, 23, or 24 hrs from each other. In some aspects, applying the alternating electric fields simultaneously with administering the one or more FGF inhibitors or FGFR inhibitors comprises applying the alternating electric fields during a time in which the one or more FGF inhibitors or FGFR inhibitors is active in a certain state in the subject's body.

In some aspects, the one or more FGF or FGFR inhibitors are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g., computed tomography-guided, during surgery or biopsy) or via inhalation.

In some aspects, the frequency of the alternating electric fields is between 50 kHz and 1 MHz. In some aspects, the frequency of the alternating electric field is 150 or 200 kHz. In some aspects, the alternating electric field can be any of the ranges described herein.

In some aspects, the alternating electric field has a field strength of between 0.1 and V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of any of those described herein.

In some aspects, the disclosed methods of treating can further comprise administering a cancer therapeutic. In some aspects, the cancer therapeutic is a known cancer therapeutic other than the FGF or FGFR inhibitor. For example, the cancer therapeutic can be, but is not limited to, chemotherapy, radiation, immunotherapy, or hormone therapy.

In some aspects, the alternating electric fields are applied before, after, or simultaneously with administering the cancer therapeutic. In some aspects, the FGF or FGFR inhibitors are applied before, after, or simultaneously with administering the cancer therapeutic. In some aspects, the one or more FGF or FGFR inhibitors and cancer therapeutic are administered simultaneously and the alternating electric fields are applied before or after the FGF or FGFR inhibitors and cancer therapeutic.

In some aspects, the steps of the disclosed methods must be performed in the order the steps are listed. For example, disclosed are methods of treating a subject in need thereof comprising first applying alternating electric fields, at a frequency for a period of time, to a target site of the subject in need thereof; and then administering one or more FGF inhibitors or FGFR inhibitors or compositions comprising one or more FGF inhibitors or FGFR inhibitors to the subject in need thereof.

Alternatively, the steps of the disclosed methods can be performed in a different order than the steps are listed. For example, disclosed are methods of treating a subject in need thereof comprising first administering one or more FGF inhibitors or FGFR inhibitors or compositions comprising one or more FGF inhibitors or FGFR inhibitors to the subject in need thereof and then applying alternating electric fields, at a frequency for a period of time, to a target site of the subject in need thereof. In some aspects, administering the one or more FGF inhibitors or FGFR inhibitors or compositions comprising one or more FGF inhibitors or FGFR inhibitors first can increase the sensitivity of cells in the subject to the alternating electric fields. In some aspects, steps of the disclosed methods can be performed in any order.

F. Methods of Causing an Effect on Cells

In some aspects, causing an effect on a cancer cell can mean increasing a cell's sensitivity to alternating electric fields, increasing cytotoxicity in a cell, or enhancing or maintaining the sensitivity to alternating electrical fields.

Disclosed are methods of increasing a cell's sensitivity to alternating electric fields comprising: applying alternating electric fields, at a frequency for a period of time, to a cell; and contacting one or more FGF inhibitors or FGFR inhibitors to the cell, thereby increasing the cell's sensitivity to the alternating electric fields.

Disclosed are methods of increasing cytotoxicity in a cell comprising: applying alternating electric fields, at a frequency for a period of time, to a cell; and contacting one or more FGF inhibitors or FGFR inhibitors to the cell, thereby increasing cytotoxicity in the cell.

Disclosed are methods of maintaining, or enhancing, sensitivity to alternating electrical fields in a cell comprising: applying alternating electric fields, at a frequency for a period of time, to a cell; and contacting one or more FGF inhibitors or FGFR inhibitors to the cell, thereby maintaining, or enhancing, sensitivity to the alternating electrical fields in the cell. In some aspects, maintaining, or enhancing, sensitivity of a cell to alternating electrical fields is the same as reducing resistance of a cell to alternating electrical fields. Thus, also disclosed are methods of reducing resistance of a cell to alternating electrical fields comprising: applying alternating electric fields, at a frequency for a period of time, to a cell; and contacting one or more FGF inhibitors or FGFR inhibitors to the cell, thereby reducing resistance of the cell to alternating electrical fields.

Disclosed are methods of maintaining, or enhancing sensitivity to alternating electrical fields in a cell comprising: applying alternating electric fields, at a frequency for a period of time, to a target site of the subject in need thereof; and administering one or more FGF inhibitors or FGFR inhibitors to the subject in need thereof, thereby maintaining, or enhancing, sensitivity to the alternating electrical fields in the cell. Thus, also disclosed are methods of reducing resistance of a cell to alternating electrical fields in a cell comprising: applying alternating electric fields, at a frequency for a period of time, to a target site of the subject in need thereof; and administering one or more FGF inhibitors or FGFR inhibitors to the subject in need thereof, thereby reducing resistance of a cell to the alternating electrical fields in the cell.

In some aspects of the methods of maintaining, or enhancing, sensitivity or methods of reducing resistance, after the step of applying alternating electric fields and prior to the step of contacting or administering FGF or FGFR inhibitors, the step of detecting an increase in FGF expression in the subject or cell is performed.

In some aspects, the methods disclosed herein can further comprise a step of applying alternating electric fields, at a frequency for a period of time, to a cell can be performed after the first round of alternating electric fields plus FGF inhibitors or FGFR inhibitors have been applied or administered to the subject or cells. For example, disclosed herein are methods of maintaining, or enhancing sensitivity to alternating electrical fields in a cell comprising: applying alternating electric fields, at a frequency for a period of time, to a target site of the subject in need thereof; and administering one or more FGF inhibitors or FGFR inhibitors to the subject in need thereof, thereby maintaining, or enhancing, sensitivity to the alternating electrical fields in the cell, further comprising applying a alternating electric fields, at a frequency for a period of time, to a target site of the subject in need thereof after the sensitivity of the cells to the alternating electrical fields has been maintained or enhanced.

In some aspects, the contacting or administering step is performed at a time where the cell's or subject's response to the alternating electric fields has decreased. In some aspects, the contacting or administering step is performed 3, 4, 5, 6, 7, 8, 9, or 10 days after applying alternating electric fields. In some aspects, the detection of an increase in FGF expression is determined prior to contacting the cell with or administering to the subject a FGF or FGF inhibitor. In some aspects, detecting FGF expression is performed prior to applying alternating electric fields to get a baseline reading and then detected again at least 1, 2, 3, 4, 5, 6, or 7 days or at least 1, 2, 3, or 4 weeks after applying alternating electric fields. In some aspects, determining whether a subject's response to the alternating electric fields has decreased can be measured by analyzing FGF downstream signaling. For example determining whether a subject's response to the alternating electric fields has decreased can be measured by determining the presence of FGF in the serum, determining the presence of pAkt in biopsies, and/or determining a lack of response in tumor reduction (or tumor growth).

In some aspects, the disclosed methods can be performed directly to a subject. For example, the methods can be performed directly to a subject in need thereof, wherein the subject is a living subject. In some aspects, the disclosed methods can be performed to a sample obtained from a subject. In some aspects, the disclosed methods can be performed outside of a subject and therefore, the cell can be in vitro. In some aspects, the disclosed methods can be performed inside of a subject and therefore, the cell can be in a subject. In some aspects, the cell (in vitro or in vivo) can be a mesothelioma cell, ovarian cancer cell, lung cancer cell, brain cancer cell, pancreatic cancer cell, breast cancer cell, or colon cancer cell. In some aspects, the cell can be from a cell line, including, but not limited to, the cell line A2780 or H1299.

In some aspects, the steps of the disclosed methods must be performed in the order the steps are listed. For example, disclosed are methods of treating a subject in need thereof comprising first applying alternating electric fields, at a frequency for a period of time, to a target site of the subject in need thereof; and then administering one or more FGF inhibitors or FGFR inhibitors or compositions comprising one or more FGF inhibitors or FGFR inhibitors to the subject in need thereof.

Alternatively, the steps of the disclosed methods can be performed in a different order than the steps are listed. For example, disclosed are methods of treating a subject in need thereof comprising first administering one or more FGF inhibitors or FGFR inhibitors or compositions comprising one or more FGF inhibitors or FGFR inhibitors to the subject in need thereof and then applying alternating electric fields, at a frequency for a period of time, to a target site of the subject in need thereof. In some aspects, administering the one or more FGF inhibitors or FGFR inhibitors or compositions comprising one or more FGF inhibitors or FGFR inhibitors first can increase the sensitivity of cells in the subject to the alternating electric fields.

In some aspects, steps of the disclosed methods can be performed in any order.

G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of FGF or FGFR inhibitors and one or more materials for delivering alternating electric fields, such as the Optune system. Materials for delivering alternating electric fields can include a signal generator and a pair of electrodes. In some embodiments, materials for delivering alternating electric fields can include a signal generator (e.g. AC signal generator), one or more pairs of electrode arrays, where each electrode array includes two or more electrodes, or a controller. In some aspects, materials for delivering alternating electric fields can include the Optune system or one or more components thereof.

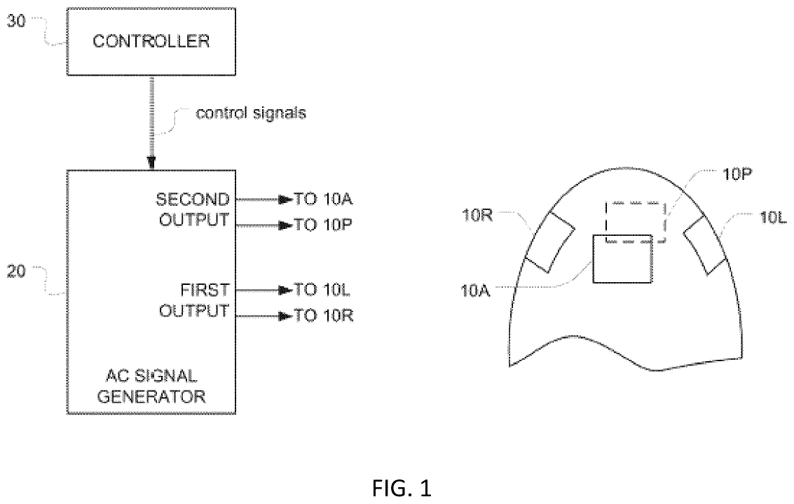

In some aspects, materials for delivering alternating electric fields can include a system comprising one or more of the elements of the system shown in FIG. 1. FIG. 1 is a block diagram of an exemplary system of materials for delivering an alternating electric field. The system includes an AC signal generator 20 that is designed to generate first and second AC outputs, for example at a frequency between 50 and 500 kHz. When the system is used to apply an alternating electric field to a person's body, the first AC output is applied across a first pair of electrodes 10L and 10R that are positioned to the left and right of the cancer cells; and the second AC output is applied across a second pair of electrodes 10A and 10P that are positioned anterior and posterior to the cancer cells. The AC signal generator 20 could also be used to apply an alternating electric field to an in vitro culture by applying the first AC output to electrodes positioned on the left and right walls of a dish (or specialized Inovitro™ dish) and applying the second AC output to electrodes positioned on the front and back walls of the dish (or specialized Inovitro™ dish). In either case, the voltages generated by the AC signal generator 20 should be sufficient to induce an electric field of at least 1 V/cm in at least a portion of the cancer cells. In some embodiments, the voltages generated by the AC signal generator 20 should be sufficient to induce an electric field of between 1 V/cm and 10 V/cm in at least a portion of the cancer cells.

In some embodiments, (a) the first AC output is applied to the L/R electrodes for a 1 second sub-interval of time; (b) the second AC output is applied to the A/P electrodes for a 1 second sub-interval of time; and the two-step sequence (a) and (b) is repeated for the duration of the treatment. The AC signal generator 20 may be configured to generate first and second AC outputs such that the first and second AC outputs have amplitudes and/or frequencies that depend on a state of at least one control input.

A controller 30 continuously sends control signals to the at least one control input during each 1 second sub-interval. Note that although FIG. 1 depicts the controller 30 and the AC signal generator 20 as two distinct blocks, those two blocks may be integrated into a single hardware device. The details of the construction of the controller 30 and the nature of the control signals will depend on the design of the AC signal generator 20. In one example, the design of the AC signal generator 20 is similar to the AC signal generator described in U.S. Pat. No. 9,910,453, which is incorporated herein by reference in its entirety. This particular AC signal generator has two output channels (i.e., a first channel for L/R and a second channel for A/P).

In some embodiments, disclosed are kits that comprise one or more of FGF and FGFR inhibitors, materials for delivering alternating electric fields, and a further therapeutic agent. For example disclosed are kits comprising one or more of aprepitant, auranofin, captopril, celecoxib, disulfiram, itraconazole, minocycline, ritonavir, sertraline, one or more FGF and FGFR inhibitors, and one or more materials for delivering alternating electric fields, such as the Optune system. In some aspects, the kits can also include Temozolomide.

### Examples

### A. Example 1

### 1. Summary

The FGF/FGFR signaling pathway plays a role in the development and progression of different cancers. The FGF signaling pathway regulates numerous cellular processes such as cell proliferation, apoptosis, angiogenesis, migration, invasion and metastasis. FGF/FGFR can be regulated by Notch, N-CAM, miRNAs, synthetic compounds, antibodies, and natural agents. FGF/FGFR exhibits its physiological functions via regulation of its downstream targets (e.g., Ras, PI3K/AKT, ERK, NF-kB, VEGF). Thus, targeting FGF/FGFR can be an effective approach for the treatment of cancer patients.

FGF/FGFR exhibits its physiological functions by regulating the main downstream signaling pathway, such as RAS/MAPK and PI3K/AKT/mTOR, FGF/FGFR can be blocked by the chemical inhibitors of FGF/FGFR, antibodies, receptor decoys and natural agents. Therefore, targeting FGF/FGFR can be an effective approach for the treatment of different cancers, such as female reproductive system cancer patients. Secreted FGFs bind to one of four transmembrane receptors with intracellular tyrosine kinase domains (FGF R1, FGF R2, FGF R3, and FGF R4) in a 2:2:2 HSPG-FGF-FGF receptor ratio. Ligand binding specificity is determined by the differential expression patterns of the FGFs, FGF receptors, and glycosaminoglycan structures, different receptor binding capacities, the requirement for specific co-factors such as the Klotho family proteins, and alternative splicing of the FGF receptors that results in two different versions of the extracellular Ig-like domain III (b or c). Following HSPG-ligand-binding to the FGF receptor, the receptor homodimerizes, leading to activation of the cytoplasmic intracellular kinase domain of the receptor, and recruitment and docking of adaptor proteins such as FRS2, GRB2, Shb, and Shc. These adaptor proteins subsequently activate multiple downstream signaling pathways including the Ras-MAPK pathway, the Jak-STAT pathway, the PI 3-Kinase-Akt pathway, the PLC gamma pathway, and the p38 and JNK MAPK pathways. Through these signaling pathways, FGFs promote fundamental cellular processes such as survival, proliferation, differentiation, and motility. Thus, targeting FGF can be helpful in treating cancer alone or in combination with other known cancer treatments, such as TTFields.

Targeting FGF/FGFR has been found to potentiate the antitumor effect of Tumor Treating Fields (TTFields).

### 2. Materials and Methods

i. Cell Culture Model

Human cell lines A2780 (ovarian carcinoma), and H1299 (non-small cell lung carcinoma) were obtained from the American Tissue Culture Collection (ATCC). Cells were grown in media supplemented with 10% (v/v) fetal bovine serum (FBS), and streptomycin (50 μg/ml) in a 37° C. humidified incubator supplied with 5% CO2. Media and supplements were purchased from Biological Industries (Beit Haemek).

ii. Mice Model

An orthotropic ovarian cancer mice model was established. MOSE-L-ffl cancer 5000/5 μl cells were injected to 12 weeks old female mice.

iii. In Vitro

a. Inovitro System

TTFields (1.7 V/cm RMS) were applied at 200 kHz to A2780 and at 150 kHz to H1229 cells using the inovitroTM system.

b. Cell Count

Cytotoxicity was determined by cell counting using Macsquant (Miltenyi Biotec) flow cytometer and presented as percentage of cells relative to control.

iv. In Vivo

Mice were treated with TTFields for 10 days using the INOVIVO system (Novocure, Haifa, Israel). Mice were sacrificed and blood samples were drawn and collected in designated blood serum tubes Centrifuged for 15 minutes in 1000 g and kept in −20° C.

v. Cytokine Array Kit Analysis and Quantification

The cytokine array kit (R&D systems ARY005B for human derived samples and ARY028 for mouse derived samples) was used to simultaneously detect 105 cytokines in each Human cell lines conditioned media (CM)/mice serum sample were collected according to the manufacturer's instructions. Array membranes were incubated for 1 hour at room temperature in blocking buffer (all reagents were supplied with the array kit). Prior to mix with reconstituted detection antibody cocktails, samples were incubated on the membranes overnight at 4° C. on a rocking platform shaker. All of the following steps were performed at room temperature, and all wash procedures involved three washes in 1× wash buffer for 10 minutes. After incubation with detection antibody, membranes were washed and incubated with streptavidin-conjugated horseradish peroxidase (1:2000) for 30 minutes on a rocking platform shaker. Unbound reagents were removed by washing, and the membranes were incubated in chemiluminescent detection reagent for 1 minute. To evaluate changes following TTFields treatment vs. the control group spots were measured for pixel density using R&D systems quantification software (quick spots) and the results were analyzed as followed: [(protein pixel density in TTFields treated sample) ÷ (protein pixel density in the control sample)].

### 3. Results

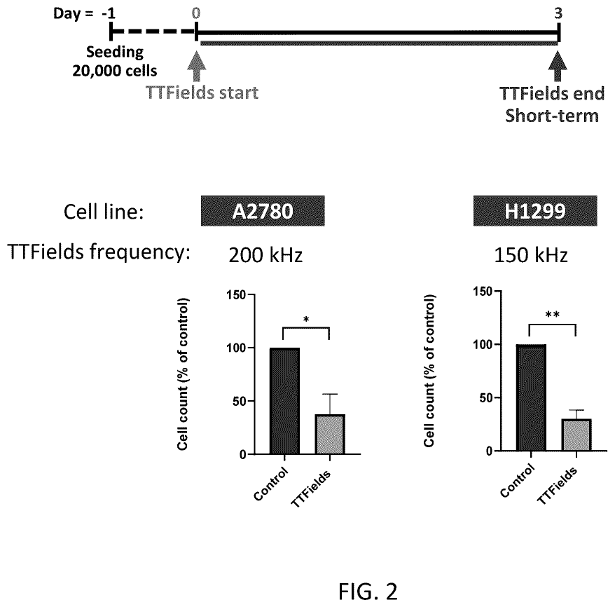

FIG. 2 shows an example of the cytotoxic effect after 3 days of TTFields treatment in Optimum cytotoxic frequencies. In order to characterize the secretory cytokine profile following TTFields application, a series of in vitro experiments were conducted in which the cells were treated for 72 hours in their cytotoxic frequencies and the cytokine array assay was used to evaluate changes in the secretory protein expression. Cell count significantly decreased in both A2780 and H1299 cells after 3 days of TTFields.

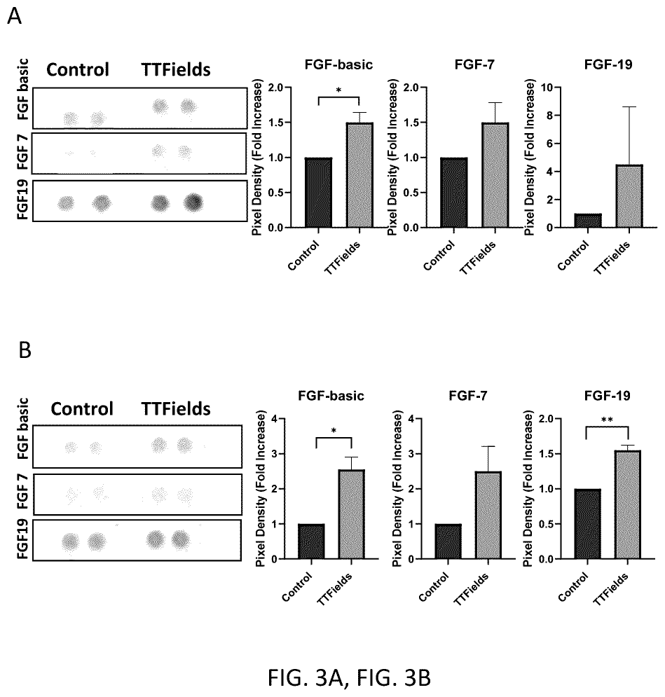

There was an up regulation in the expression of the FGF family growth factors, particularly FGF-basic, FGF-7, and FGF-19, after 3 days of treatment of TTFields in optimal cytotoxic frequencies for A2780 (200 kHz) and H1299 (150 kHz) cell lines (See FIGS. 3A and 3B).

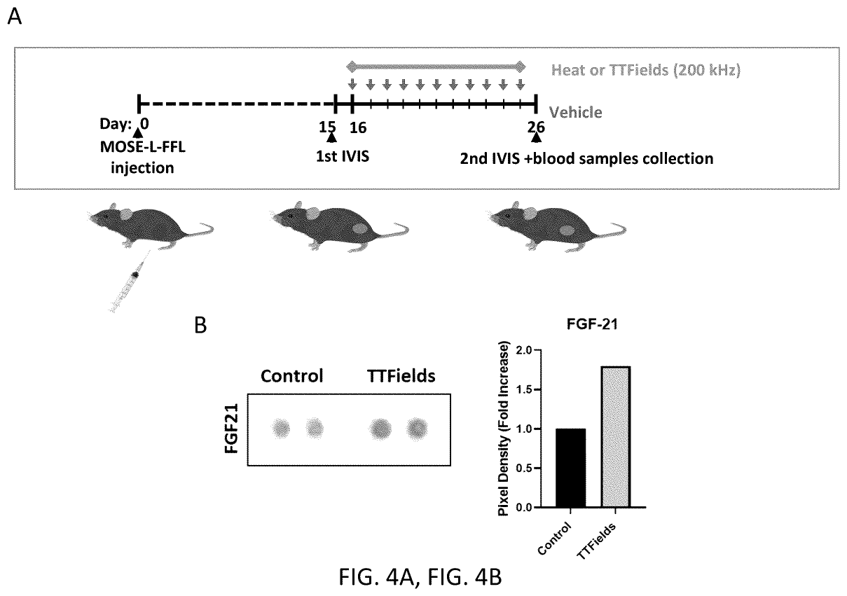

FIG. 4 shows that FGF-21 expression is upregulated following 10 days of TTFields application in an ovarian cancer mouse model.

Based on these results, one can inhibit/block FGF/FGFR signaling in order to enhance or maintain the effectiveness of TTFields while decreasing the negative effects of FGF/FGFR signaling.

### B. Example 2

### 1. Background

Glioblastoma (GBM) is an aggressive primary brain tumor with poor outcome despite the standard treatment associating radiotherapy and temozolomide according to the Stupp protocol. Recently, a novel therapy termed tumor treating fields (TTFields) has been approved for clinical treatment as both monotherapy for recurrent GBM and in combination with adjuvant post-chemoradiation. Moreover, it has been shown that TTFields act in additive or synergistic manner with irradiation and pilot clinical study has shown good tolerance of this combination allowing the development of randomized Trident trial. However even with the adjunction of TTFields, GBM remains a lethal disease with resistance to treatment. One of the causes of resistance of GBM is the presence of cancer stem cells (GSC). This pool of cells is responsible for tumor cell resistance to therapies and subsequent tumor-initiating cell proliferation that favors tumor regrowth. Their maintenance is favored by hypoxia. Studying mechanisms of resistance of GSC to TTFields is a major issue to address.

FGFR1 has been demonstrated to control glioblastoma and glioblastoma stem cells radioresistance (Gouaze-Andersson et al., Cancer Res.2016; Gouaze-Andersson et al. Oncotarget 2018) and that its inhibition with FGFR inhibitor led to glioblastoma radiosensitization (Ader et al, Eur. J. Cancer 2014). Moreover, Clinical data demonstrates that GBM patients with high levels of FGFR1 have a shorter time to progression as well as a shorter overall survival after radiotherapy (Ducassou et al, Eur J Cancer 2013) and that GBM patients expressing in the tumor the five genes signature, including a6-integrin, ZEB1/YAP1, FGFR1 and FOXMl, have a significantly shorter overall survival (Kowalski-Chauvel et al, Cancers 2019 11(3)). These data indicate that targeting FGFR1 can increase the efficacy of glioblastoma treatment. In this study, GBM stem cells' resistance mechanisms to TTFields, and more particularly on FGFR1 pathway, are examined.

### 2. Research Design and Methods

This study focuses on the involvement of FGFR1 in the control of sensitivity to TTFields. The study was performed on several glioblastoma stem cells (GSC) fully characterized in vitro and in vivo that were obtained from clinical trial STEMRI (NCT01872221) at Institut Claudius Regaud. The inventors wish to thank Institut Claudius Regaud for providing the glioblastoma stem cells used in these experiments.

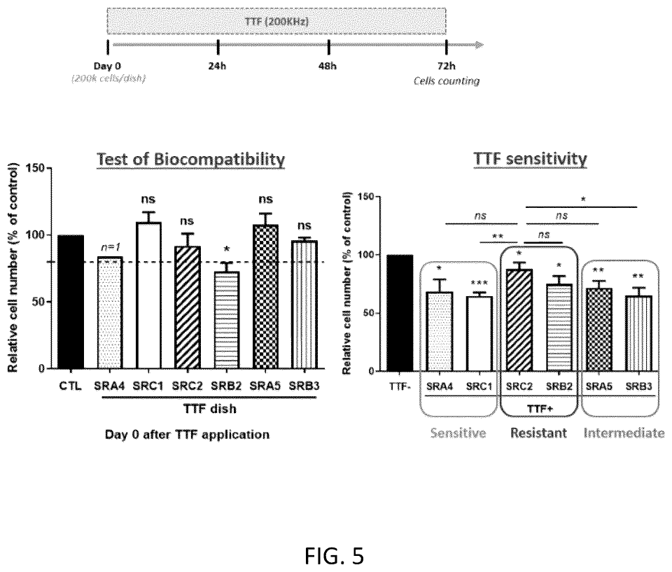

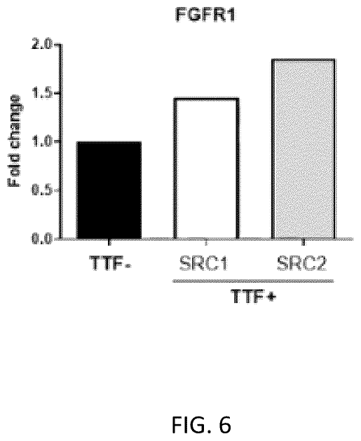

FIG. 5 shows that TTFields treatment induce different cytotoxic effect levels that can discriminate 3 groups of GSC such as sensitive, intermediate and resistant to TTFields. FIG. 6 shows that FGFR1 RNA expression is increased by TTFields in GSCs. FGFR1 expression was increased in both TTF sensitive and resistant GSC.

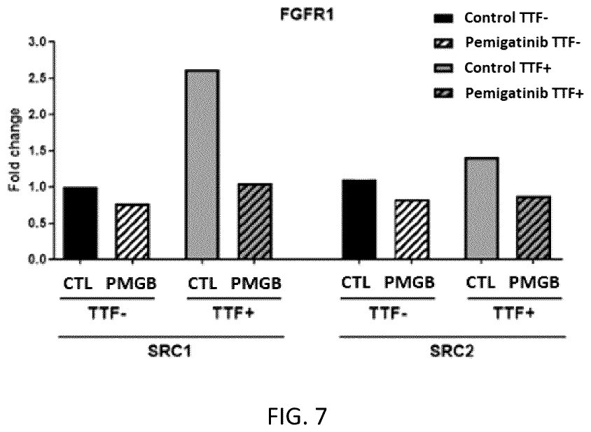

FIG. 7 shows expression levels of FGFR1 in response to TTFields in resistant (SRC2) and sensitive (SRC1) cells and its inhibition using an FGFR1 inhibitor, pemigatinib.

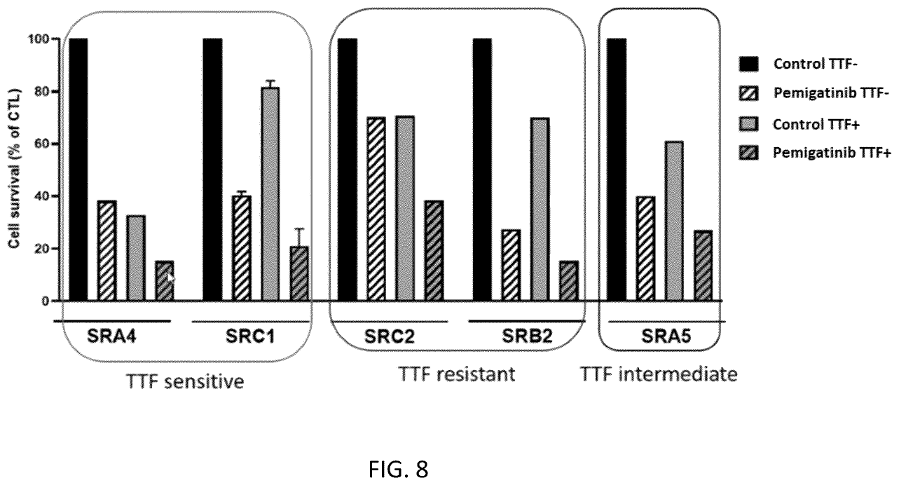

FIG. 8 shows that FGFR1 inhibitors can sensitize all glioblastoma cell lines to TTFields including cells that were previously resistant to TTFields. The mechanism of radiation to induce cell death can be a result of DNA damage which leads to apoptosis, because it is known that TTFields also generate DNA damage and reduce the DNA repair mechanism. Radio sensitization is reduced following addition of FGFR inhibitor. There is increased sensitivity to TTFields when there is concomitant treatment with FGFR inhibition which indicates a synergistic result of increased secretion of FGF family members in serum and conditioned media following TTFields application, thus explaining the reduced sensitivity of cells to TTFields.

These results discriminate GSC sensitive versus resistant to TTFields and show that inhibition of FGFR1 can increase sensitivity of glioblastoma cells to TTFields.

### Illustrative Embodiments

One example of the many embodiments described herein is a method of treating a subject in need thereof comprising applying alternating electric fields, at a frequency for a period of time, to a target site of the subject in need thereof; and administering one or more fibroblast growth factor (FGF) inhibitors or fibroblast growth factor receptor (FGFR) inhibitors to the subject in need thereof.

In one example of the many embodiments described herein the subject is living.

In one example of the many embodiments described herein the subject has mesothelioma, ovarian cancer or lung cancer.

In one example of the many embodiments described herein the target site comprises one or more cancer cells.

In one example of the many embodiments described herein the alternating electric fields are applied before, after, or simultaneously with administering the one or more FGF or FGFR inhibitors.

In one example of the many embodiments described herein the one or more FGF or FGFR inhibitors are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation.

One example of the many embodiments described herein is a method of increasing a cell's sensitivity to alternating electric fields comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing the cell's sensitivity to the alternating electric fields.

One example of the many embodiments described herein is a method of increasing a cell's sensitivity to alternating electric fields comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing the cell's sensitivity to the alternating electric fields, wherein the cell is in a subject.

One example of the many embodiments described herein is a method of increasing a cell's sensitivity to alternating electric fields comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing the cell's sensitivity to the alternating electric fields, wherein the method is performed on a cell in vitro.

One example of the many embodiments described herein is a method of increasing cytotoxicity in a cell comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing cytotoxicity in the cell.

One example of the many embodiments described herein is a method of increasing cytotoxicity in a cell comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing cytotoxicity in the cell, wherein the cell is in a subject.

One example of the many embodiments described herein is a method of increasing cytotoxicity in a cell comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing cytotoxicity in the cell, wherein the method is performed on a cell in vitro.

In one example of the many embodiments described herein the FGF inhibitor inhibits or decreases FGF expression.

In one example of the many embodiments described herein the FGF inhibitor blocks upregulation of FGF expression in response to alternating electric fields.

In one example of the many embodiments described herein the FGF inhibitor prevents FGF from interacting or binding to one or more FGF receptors (FGFR).

In one example of the many embodiments described herein the FGFR inhibitor prevents FGFR from interacting or binding with one or more FGFs.

In one example of the many embodiments described herein the FGFR inhibitor blocks one or more FGF receptors (FGFR).

In one example of the many embodiments described herein the FGF is FGF-21, FGF-19, FGF-7, FGF-basic.

In one example of the many embodiments described herein the FGFR is FGFR1, FGFR2, FGFR3, or FGFR4.

In one example of the many embodiments described herein the FGFR inhibitor can be one or more of the inhibitors of Table 1.

In one example of the many embodiments described herein the cancer cell is an ovarian cancer or lung cancer cell.

In one example of the many embodiments described herein the frequency of the alternating electric field is between 50 kHz and 1 MHz.

In one example of the many embodiments described herein the frequency of the alternating electric field is 150 or 250 kHz.

In one example of the many embodiments described herein the alternating electric field has a field strength of between 0.5 and 4 V/cm RMS.

In one example of the many embodiments described herein the alternating electric field has a field strength of 0.9 V/cm RMS.

In one example of the many embodiments described herein the methods further comprising administering a cancer therapeutic.

One example of the many embodiments described herein is a method of maintaining sensitivity to an alternating electrical field in a cell comprising: applying alternating electric fields, at a frequency for a period of time, to a cell; and contacting one or more FGF inhibitors or a FGFR inhibitors to the cell, thereby maintaining sensitivity to the alternating electrical field in the cell.

One example of the many embodiments described herein is a method of maintaining sensitivity to an alternating electrical field in a cell comprising: applying alternating electric fields, at a frequency for a period of time, to a cell; and contacting one or more FGF inhibitors or a FGFR inhibitors to the cell, thereby maintaining sensitivity to the alternating electrical field in the cell, wherein the cell is in a subject.

One example of the many embodiments described herein is a method of maintaining sensitivity to an alternating electrical field in a cell comprising: applying alternating electric fields, at a frequency for a period of time, to a cell; and contacting one or more FGF inhibitors or a FGFR inhibitors to the cell, thereby maintaining sensitivity to the alternating electrical field in the cell, wherein the method is performed on a cell in vitro.

One example of the many embodiments described herein is a method of maintaining sensitivity to an alternating electrical field in a subject applying alternating electric fields, at a frequency for a period of time, to a target site of the subject in need thereof; and administering one or more fibroblast growth factor (FGF) inhibitors or a fibroblast growth factor receptor (FGFR) inhibitors to the subject in need thereof, thereby maintaining sensitivity to the alternating electrical field in the cell.

One example of the many embodiments described herein is a method of maintaining sensitivity to an alternating electrical field in a cell applying alternating electric fields, at a frequency for a period of time, to a target site of the subject in need thereof; and administering one or more fibroblast growth factor (FGF) inhibitors or a fibroblast growth factor receptor (FGFR) inhibitors to the subject in need thereof, thereby maintaining sensitivity to the alternating electrical field in the cell.

In one example of the many embodiments described herein described herein is a method of maintaining sensitivity to an alternating electrical field in a subject applying alternating electric fields, at a frequency for a period of time, to a target site of the subject in need thereof; and administering one or more fibroblast growth factor (FGF) inhibitors or a fibroblast growth factor receptor (FGFR) inhibitors to the subject in need thereof, thereby maintaining sensitivity to the alternating electrical field in the subject or cell, wherein the subject is living.

In one example of the many embodiments described herein the methods further comprise, after applying alternating electric fields and prior to administering one or more fibroblast growth factor (FGF) inhibitors or a fibroblast growth factor receptor (FGFR) inhibitors to the subject in need thereof, detecting an increase in FGF expression in the subject or cell.

In one example of the many embodiments described herein the contacting is performed at a time where the cell's response to the alternating electric fields has decreased.

In one example of the many embodiments described herein the contacting one or more fibroblast growth factor (FGF) inhibitors or a fibroblast growth factor receptor (FGFR) inhibitors is performed 3, 4, 5, 6, 7, 8, 9, or 10 days after applying the alternating electric fields is performed.

In one example of the many embodiments described herein the contacting is performed at a time where the subject's response to the alternating electric fields has decreased.

In one example of the many embodiments described herein the contacting is performed 3, 4, 5, 6, 7, 8, 9, or 10 days after step a) is performed.

In one example of the many embodiments described herein the alternating electric fields are applied before, after, or simultaneously with administering the one or more FGF or FGFR inhibitors.

In one example of the many embodiments described herein the one or more FGF or FGFR inhibitors are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation.

In one example of the many embodiments described herein the FGF inhibitor inhibits or decreases FGF expression.

In one example of the many embodiments described herein the FGF inhibitor blocks upregulation of FGF expression in response to alternating electric fields.

In one example of the many embodiments described herein the FGF inhibitor prevents FGF from interacting or binding to one or more FGF receptors (FGFR).

In one example of the many embodiments described herein the FGFR inhibitor prevents FGFR from interacting or binding with one or more FGFs.

In one example of the many embodiments described herein the FGFR inhibitor blocks one or more FGF receptors (FGFR).

In one example of the many embodiments described herein the FGF is FGF-21, FGF-19, FGF-7, or FGF-basic.

In one example of the many embodiments described herein the FGFR is FGFR1, FGFR2, FGFR3, or FGFR4.

In one example of the many embodiments described herein the FGFR inhibitor can be one or more of the inhibitors of Table 1.

In one example of the many embodiments described herein the frequency of the alternating electric field is between 50 kHz and 1 MHz.

In one example of the many embodiments described herein the frequency of the alternating electric field is 150 or 250 kHz.

In one example of the many embodiments described herein the alternating electric field has a field strength of between 0.5 and 4 V/cm RMS.

In one example of the many embodiments described herein the alternating electric field has a field strength of 0.9 V/cm RMS.

In one example of the many embodiments described herein the methods further comprise administering a cancer therapeutic.

One example of the many embodiments described herein is a fibroblast growth factor (FGF) inhibitor or fibroblast growth factor receptor (FGFR) inhibitor for use in a method of treating a subject in need thereof, the method comprising applying alternating electric fields, at a frequency for a period of time, to a target site of the subject in need thereof; and administering a fibroblast growth factor (FGF) inhibitor or fibroblast growth factor receptor (FGFR) inhibitor to the subject in need thereof.

One example of the many embodiments described herein is a fibroblast growth factor (FGF) inhibitor or fibroblast growth factor receptor (FGFR) inhibitor for use in a method of increasing a cell's sensitivity to alternating electric fields comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing the cell's sensitivity to the alternating electric fields.

One example of the many embodiments described herein is a fibroblast growth factor (FGF) inhibitor or fibroblast growth factor receptor (FGFR) inhibitor for use in a method of increasing a cell's sensitivity to alternating electric fields comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing the cell's sensitivity to the alternating electric fields, wherein the cell is in a subject.

One example of the many embodiments described herein is a fibroblast growth factor (FGF) inhibitor or fibroblast growth factor receptor (FGFR) inhibitor for use in a method of increasing a cell's sensitivity to alternating electric fields comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing the cell's sensitivity to the alternating electric fields, wherein the method is performed on a cell in vitro.

One example of the many embodiments described herein is a fibroblast growth factor (FGF) inhibitor or fibroblast growth factor receptor (FGFR) inhibitor for use in a method of increasing cytotoxicity in a cell comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing cytotoxicity in the cell.

One example of the many embodiments described herein is a fibroblast growth factor (FGF) inhibitor or fibroblast growth factor receptor (FGFR) inhibitor for use in a method of increasing cytotoxicity in a cell comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing cytotoxicity in the cell, wherein the cell is in a subject.

One example of the many embodiments described herein is a fibroblast growth factor (FGF) inhibitor or fibroblast growth factor receptor (FGFR) inhibitor for use in a method of increasing cytotoxicity in a cell comprising: applying alternating electric fields, at a frequency for a period of time, to a cell; and contacting one or more fibroblast growth factor (FGF) inhibitors or a fibroblast growth factor receptor (FGFR) inhibitors to the cell, thereby increasing cytotoxicity in the cell, wherein the method is performed on a cell in vitro.

In one example of the many embodiments described herein the subject is living.

In one example of the many embodiments described herein the subject has mesothelioma, ovarian cancer or lung cancer.

In one example of the many embodiments described herein the target site comprises one or more cancer cells.

In one example of the many embodiments described herein the alternating electric fields are applied before, after, or simultaneously with administering the one or more FGF or FGFR inhibitors.

In one example of the many embodiments described herein the one or more FGF or FGFR inhibitors are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation.

In one example of the many embodiments described herein the FGF inhibitor inhibits or decreases FGF expression.

In one example of the many embodiments described herein the FGF inhibitor blocks upregulation of FGF expression in response to alternating electric fields.

In one example of the many embodiments described herein the FGF inhibitor prevents FGF from interacting or binding to one or more FGF receptors (FGFR).

In one example of the many embodiments described herein the FGFR inhibitor prevents FGFR from interacting or binding with one or more FGFs.

In one example of the many embodiments described herein the FGFR inhibitor blocks one or more FGF receptors (FGFR).

In one example of the many embodiments described herein the FGF is FGF-21, FGF-19, FGF-7, FGF-basic.

In one example of the many embodiments described herein the FGFR is FGFR1, FGFR2, FGFR3, or FGFR4.

In one example of the many embodiments described herein the FGFR inhibitor can be one or more of the inhibitors of Table 1.

In one example of the many embodiments described herein the cancer cell is an ovarian cancer or lung cancer cell.

In one example of the many embodiments described herein the frequency of the alternating electric field is between 50 kHz and 1 MHz.

In one example of the many embodiments described herein the frequency of the alternating electric field is 150 or 250 kHz.

In one example of the many embodiments described herein the alternating electric field has a field strength of between 0.5 and 4 V/cm RMS.

In one example of the many embodiments described herein the alternating electric field has a field strength of 0.9 V/cm RMS.

In one example of the many embodiments described herein the methods further comprising administering a cancer therapeutic.

One example of the many embodiments described herein is a fibroblast growth factor (FGF) inhibitor or fibroblast growth factor receptor (FGFR) inhibitor for use in a method of maintaining sensitivity to an alternating electrical field in a cell comprising: applying alternating electric fields, at a frequency for a period of time, to a cell; and contacting one or more FGF inhibitors or a FGFR inhibitors to the cell, thereby maintaining sensitivity to the alternating electrical field in the cell.

One example of the many embodiments described herein is a fibroblast growth factor (FGF) inhibitor or fibroblast growth factor receptor (FGFR) inhibitor for use in a method of maintaining sensitivity to an alternating electrical field in a cell comprising: applying alternating electric fields, at a frequency for a period of time, to a cell; and contacting one or more FGF inhibitors or a FGFR inhibitors to the cell, thereby maintaining sensitivity to the alternating electrical field in the cell, wherein the cell is in a subject.

One example of the many embodiments described herein is a fibroblast growth factor (FGF) inhibitor or fibroblast growth factor receptor (FGFR) inhibitor for use in a method of maintaining sensitivity to an alternating electrical field in a cell comprising: applying alternating electric fields, at a frequency for a period of time, to a cell; and contacting one or more FGF inhibitors or a FGFR inhibitors to the cell, thereby maintaining sensitivity to the alternating electrical field in the cell, wherein the method is performed on a cell in vitro.

One example of the many embodiments described herein is a fibroblast growth factor (FGF) inhibitor or fibroblast growth factor receptor (FGFR) inhibitor for use in a method of maintaining sensitivity to an alternating electrical field in a subject applying alternating electric fields, at a frequency for a period of time, to a target site of the subject in need thereof; and administering one or more fibroblast growth factor (FGF) inhibitors or a fibroblast growth factor receptor (FGFR) inhibitors to the subject in need thereof, thereby maintaining sensitivity to the alternating electrical field in the cell.

One example of the many embodiments described herein is a fibroblast growth factor (FGF) inhibitor or fibroblast growth factor receptor (FGFR) inhibitor for use in a method of maintaining sensitivity to an alternating electrical field in a cell applying alternating electric fields, at a frequency for a period of time, to a target site of the subject in need thereof; and administering one or more fibroblast growth factor (FGF) inhibitors or a fibroblast growth factor receptor (FGFR) inhibitors to the subject in need thereof, thereby maintaining sensitivity to the alternating electrical field in the cell.

One example of the many embodiments described herein is a fibroblast growth factor (FGF) inhibitor or fibroblast growth factor receptor (FGFR) inhibitor for use in a method of maintaining sensitivity to an alternating electrical field in a subject applying alternating electric fields, at a frequency for a period of time, to a target site of the subject in need thereof; and administering one or more fibroblast growth factor (FGF) inhibitors or a fibroblast growth factor receptor (FGFR) inhibitors to the subject in need thereof, thereby maintaining sensitivity to the alternating electrical field in the subject or cell, wherein the subject is living.

In one example of the many embodiments described herein the methods further comprise, after applying alternating electric fields and prior to administering one or more fibroblast growth factor (FGF) inhibitors or a fibroblast growth factor receptor (FGFR) inhibitors to the subject in need thereof, detecting an increase in FGF expression in the subject or cell.

In one example of the many embodiments described herein the contacting is performed at a time where the cell's response to the alternating electric fields has decreased.

In one example of the many embodiments described herein the contacting one or more fibroblast growth factor (FGF) inhibitors or a fibroblast growth factor receptor (FGFR) inhibitors is performed 3, 4, 5, 6, 7, 8, 9, or 10 days after applying the alternating electric fields is performed.

In one example of the many embodiments described herein the contacting is performed at a time where the subject's response to the alternating electric fields has decreased.

In one example of the many embodiments described herein the contacting is performed 3, 4, 5, 6, 7, 8, 9, or 10 days after step a) is performed.

In one example of the many embodiments described herein the alternating electric fields are applied before, after, or simultaneously with administering the one or more FGF or FGFR inhibitors.

In one example of the many embodiments described herein the one or more FGF or FGFR inhibitors are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation.

In one example of the many embodiments described herein the FGF inhibitor inhibits or decreases FGF expression.

In one example of the many embodiments described herein the FGF inhibitor blocks upregulation of FGF expression in response to alternating electric fields.

In one example of the many embodiments described herein the FGF inhibitor prevents FGF from interacting or binding to one or more FGF receptors (FGFR).

In one example of the many embodiments described herein the FGFR inhibitor prevents FGFR from interacting or binding with one or more FGFs.

In one example of the many embodiments described herein the FGFR inhibitor blocks one or more FGF receptors (FGFR).

In one example of the many embodiments described herein the FGF is FGF-21, FGF-19, FGF-7, or FGF-basic.

In one example of the many embodiments described herein the FGFR is FGFR1, FGFR2, FGFR3, or FGFR4.

In one example of the many embodiments described herein the FGFR inhibitor can be one or more of the inhibitors of Table 1.

In one example of the many embodiments described herein the frequency of the alternating electric field is between 50 kHz and 1 MHz.

In one example of the many embodiments described herein the frequency of the alternating electric field is 150 or 250 kHz.

In one example of the many embodiments described herein the alternating electric field has a field strength of between 0.5 and 4 V/cm RMS.

In one example of the many embodiments described herein the alternating electric field has a field strength of 0.9 V/cm RMS.

In one example of the many embodiments described herein the methods further comprise administering a cancer therapeutic.

One example of the many embodiments described herein is a combination of alternating electric fields and one or more fibroblast growth factor (FGF) inhibitors or fibroblast growth factor receptor (FGFR) inhibitors for use in the treatment of a subject in need thereof.

In one example of the many embodiments described herein the alternating electric fields are applied at a frequency for a period of time to a target site of the subject in need thereof.

In one example of the many embodiments described herein the subject has mesothelioma, ovarian cancer or lung cancer.

In one example of the many embodiments described herein the target site comprises one or more cancer cells.

In one example of the many embodiments described herein the alternating electric fields are applied before, after, or simultaneously with administering the one or more FGF or FGFR inhibitors.

In one example of the many embodiments described herein the one or more FGF or FGFR inhibitors are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation.

One example of the many embodiments described herein is a combination of alternating electric fields and one or more fibroblast growth factor (FGF) inhibitors or fibroblast growth factor receptor (FGFR) inhibitors for use in increasing a cell's sensitivity to alternating electric fields.

One example of the many embodiments described herein is a combination of alternating electric fields and one or more fibroblast growth factor (FGF) inhibitors or fibroblast growth factor receptor (FGFR) inhibitors for use in increasing a cell's sensitivity to alternating electric fields, wherein the cell is in a subject.

One example of the many embodiments described herein is a combination of alternating electric fields and one or more fibroblast growth factor (FGF) inhibitors or fibroblast growth factor receptor (FGFR) inhibitors for use in increasing a cell's sensitivity to alternating electric fields, wherein the method is performed on a cell in vitro.

One example of the many embodiments described herein is a combination of alternating electric fields and one or more fibroblast growth factor (FGF) inhibitors or fibroblast growth factor receptor (FGFR) inhibitors for use in increasing cytotoxicity in a cell.

One example of the many embodiments described herein is a combination of alternating electric fields and one or more fibroblast growth factor (FGF) inhibitors or fibroblast growth factor receptor (FGFR) inhibitors for use in increasing cytotoxicity in a cell, wherein the cell is in a subject.

One example of the many embodiments described herein is a combination of alternating electric fields and one or more fibroblast growth factor (FGF) inhibitors or fibroblast growth factor receptor (FGFR) inhibitors for use in increasing cytotoxicity in a cell, wherein the method is performed on a cell in vitro.

In one example of the many embodiments described herein the alternating electric fields are applied at a frequency for a period of time to the cell.

In one example of the many embodiments described herein the one or more FGF inhibitors or FGFR inhibitors are contacted with the cell.

In one example of the many embodiments described herein the alternating electric fields are applied before, after, or simultaneously with administering the one or more FGF or FGFR inhibitors.

In one example of the many embodiments described herein the preceding embodiments, wherein the one or more FGF or FGFR inhibitors are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation.

In one example of the many embodiments described herein the cell is in vitro.

In one example of the many embodiments described herein the cell is in a subject.

In one example of the many embodiments described herein the cancer cell is an ovarian cancer or lung cancer cell.

One example of the many embodiments described herein is a combination of alternating electric fields and one or more fibroblast growth factor (FGF) inhibitors or fibroblast growth factor receptor (FGFR) inhibitors for use in maintaining sensitivity to an alternating electrical field in a cell.

One example of the many embodiments described herein is a combination of alternating electric fields and one or more fibroblast growth factor (FGF) inhibitors or fibroblast growth factor receptor (FGFR) inhibitors for use in maintaining sensitivity to an alternating electrical field in a cell, wherein the cell is in a subject.

One example of the many embodiments described herein is a combination of alternating electric fields and one or more fibroblast growth factor (FGF) inhibitors or fibroblast growth factor receptor (FGFR) inhibitors for use in maintaining sensitivity to an alternating electrical field in a cell, wherein the method is performed on a cell in vitro.

One example of the many embodiments described herein is a combination of alternating electric fields and one or more fibroblast growth factor (FGF) inhibitors or fibroblast growth factor receptor (FGFR) inhibitors for use in maintaining sensitivity to an alternating electrical field in a subject.

One example of the many embodiments described herein is a combination of alternating electric fields and one or more fibroblast growth factor (FGF) inhibitors or fibroblast growth factor receptor (FGFR) inhibitors for use in maintaining sensitivity to an alternating electrical field in a subject, wherein the cell is in a subject.

One example of the many embodiments described herein is a combination of alternating electric fields and one or more fibroblast growth factor (FGF) inhibitors or fibroblast growth factor receptor (FGFR) inhibitors for use in maintaining sensitivity to an alternating electrical field in a subject, wherein the method is performed on a cell in vitro.

In one example of the many embodiments described herein the FGF expression is detected in the subject or cell.

In one example of the many embodiments described herein the cell's or subject's response to the alternating electric fields decreases before use of the one or more FGF inhibitors or FGFR inhibitors.

In one example of the many embodiments described herein the use of the one or more FGF inhibitors or FGFR inhibitors occurs 3, 4, 5, 6, 7, 8, 9, or 10 days after applying the alternating electric fields.

In one example of the many embodiments described herein the alternating electric fields are applied before, after, or simultaneously with administering the one or more FGF or FGFR inhibitors.

In one example of the many embodiments described herein the one or more FGF or

FGFR inhibitors are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation.

In one example of the many embodiments described herein the FGF inhibitor inhibits or decreases FGF expression.

In one example of the many embodiments described herein the FGF inhibitor blocks upregulation of FGF expression in response to alternating electric fields.

In one example of the many embodiments described herein the FGF inhibitor prevents FGF from interacting or binding to one or more FGF receptors (FGFR).

In one example of the many embodiments described herein the FGFR inhibitor prevents FGFR from interacting or binding with one or more FGFs.

In one example of the many embodiments described herein the FGFR inhibitor blocks one or more FGF receptors (FGFR).

In one example of the many embodiments described herein the FGF is FGF-21, FGF-19, FGF-7, or FGF-basic.

In one example of the many embodiments described herein the FGFR is FGFR1, FGFR2, FGFR3, or FGFR4.

In one example of the many embodiments described herein the FGFR inhibitor can be one or more of the inhibitors of Table 1.

In one example of the many embodiments described herein the frequency of the alternating electric field is between 50 kHz and 1 MHz.

In one example of the many embodiments described herein the frequency of the alternating electric field is 150 or 250 kHz.

In one example of the many embodiments described herein the alternating electric field has a field strength of between 0.5 and 4 V/cm RMS.

In one example of the many embodiments described herein the alternating electric field has a field strength of 0.9 V/cm RMS.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating a subject in need thereof comprising:
a) applying alternating electric fields, at a frequency for a period of time, to a target site of the subject in need thereof; and
b) administering one or more fibroblast growth factor (FGF) inhibitors or fibroblast growth factor receptor (FGFR) inhibitors to the subject in need thereof.

2. The method of claim 1, wherein the subject has mesothelioma, ovarian cancer or lung cancer.

3. The method of claim 1, wherein the target site comprises one or more cancer cells.

4. The method of claim 1, wherein the alternating electric fields are applied before, after, or simultaneously with administering the one or more FGF or FGFR inhibitors.

5. The method of claim 1, wherein the one or more FGF or FGFR inhibitors are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g. computed tomography-guided, during surgery or biopsy) or via inhalation.

6. A method of increasing a cell's sensitivity to alternating electric fields comprising:
a) applying alternating electric fields, at a frequency for a period of time, to a cell; and
b) contacting one or more fibroblast growth factor (FGF) inhibitors or a fibroblast growth factor receptor (FGFR) inhibitors to the cell,
thereby increasing the cell's sensitivity to the alternating electric fields.

7. A method of increasing cytotoxicity in a cell comprising:
a) applying alternating electric fields, at a frequency for a period of time, to a cell; and
b) contacting one or more fibroblast growth factor (FGF) inhibitors or a fibroblast growth factor receptor (FGFR) inhibitors to the cell,
thereby increasing cytotoxicity in the cell.

8. The method of claim 6, wherein the cell is in vitro or in a subject.

9. The method of claim 1, wherein the FGF inhibitor inhibits or decreases FGF expression.

10. The method of claim 1, wherein the FGF inhibitor blocks upregulation of FGF expression in response to alternating electric fields.

11. The method of claim 1, wherein the FGF inhibitor prevents FGF from interacting or binding to one or more FGF receptors (FGFR).

12. The method of claim 1, wherein the FGFR inhibitor prevents FGFR from interacting or binding with one or more FGFs.

13. The method of claim 1, wherein the FGFR inhibitor blocks one or more FGF receptors (FGFR).

14. The method of claim 1, wherein the FGF is FGF-21, FGF-19, FGF-7, FGF-basic.

15. The method of claim 1, wherein the FGFR is FGFR1, FGFR2, FGFR3, or FGFR4.

16. The method of claim 1, wherein the FGFR inhibitor can be one or more of the inhibitors of Table 1.

17. The method of claim 6, wherein the cancer cell is an ovarian cancer or lung cancer cell.

18. The method of claim 1, wherein the frequency of the alternating electric field is between 50 kHz and 1 MHz.

19. The method of claim 1, wherein the alternating electric field has a field strength of between 0.5 and 4 V/cm RMS.

20. The method of claim 1, further comprising administering a cancer therapeutic.
